SEQUENCE LISTING (14 sequences)

SEQ ID NO: 1 - protein, 37 aa, Homo sapiens
HDEFERHAEGTFTSDVSSYLEGQAAKEFIAWLVKGRG

SEQ ID NO: 2 - protein, 31 aa, Homo sapiens
HAEGTFTSDVSSYLEGQAAKEFIAWLVKGRG

SEQ ID NO: 3 - protein, 30 aa, Homo sapiens
HAEGTFTSDVSSYLEGQAAKEFIAWLVKGR

SEQ ID NO: 4 - protein, 28 aa, Homo sapiens
EGTFTSDVSSYLEGQAAKEFIAWLVKGR

SEQ ID NO: 5 - protein, 30 aa, Artificial Sequence; identical to SEQ ID NO: 3
  Description of Artificial Sequence Synthetic polypeptide
  MOD_RES (20)..(20): Lys(Mal-PEG-BP)

SEQ ID NO: 6 - protein, 30 aa, Artificial Sequence; identical to SEQ ID NO: 3
  Description of Artificial Sequence Synthetic polypeptide
  MOD_RES (30)..(30): Arg(Mal-PEG)

SEQ ID NO: 7 - protein, 30 aa, Artificial Sequence; identical to SEQ ID NO: 3
  Description of Artificial Sequence Synthetic polypeptide
  MOD_RES (30)..(30): Arg(Mal-PEG-BP)

SEQ ID NO: 8 - protein, 30 aa, Artificial Sequence; identical to SEQ ID NO: 3
  Description of Artificial Sequence Synthetic polypeptide
  MOD_RES (28)..(28): Lys(Mal-PEG)

SEQ ID NO: 9 - protein, 30 aa, Artificial Sequence; identical to SEQ ID NO: 3
  Description of Artificial Sequence Synthetic polypeptide
  MOD_RES (28)..(28): Lys(Mal-PEG-BP)

SEQ ID NO: 10 - protein, 30 aa, Artificial Sequence; identical to SEQ ID NO: 3
  Description of Artificial Sequence Synthetic polypeptide
  MOD_RES (20)..(20): Lys(Mal-PEG)

SEQ ID NO: 11 - protein, 30 aa, Artificial Sequence; identical to SEQ ID NO: 3
  Description of Artificial Sequence Synthetic polypeptide
  MOD_RES (20)..(20): Lys(Mal-PEG)
  MOD_RES (26)..(26): Leu(Mal-PEG)

SEQ ID NO: 12 - protein, 30 aa, Artificial Sequence; identical to SEQ ID NO: 3
  Description of Artificial Sequence Synthetic polypeptide
  MOD_RES (20)..(20): Lys(Mal-PEG-BP)
  MOD_RES (26)..(26): Leu(Mal-PEG-BP)

SEQ ID NO: 13 - protein, 30 aa, Artificial Sequence; identical to SEQ ID NO: 3
  Description of Artificial Sequence Synthetic polypeptide
  MOD_RES (20)..(20): Lys(Mal-PEG)
  MOD_RES (26)..(26): Leu(Mal-PEG)
  MOD_RES (30)..(30): Arg(Mal-PEG)

SEQ ID NO: 14 - protein, 30 aa, Artificial Sequence; identical to SEQ ID NO: 3
  Description of Artificial Sequence Synthetic polypeptide
  MOD_RES (20)..(20): Lys(Mal-PEG-BP)
  MOD_RES (26)..(26): Leu(Mal-PEG-BP)
  MOD_RES (30)..(30): Arg(Mal-PEG-BP)

(19) United States
(12) Patent Application Publication
(10) Pub. No.: US 2021/0187077 A1
(43) Pub. Date: Jun. 24, 2021

(54) GLP-1 Agonist Conjugates for Sustained Glycemic Control

(71) Applicant: IDAHO STATE UNIVERSITY, POCATELLO, ID (US)

(72) Inventor: ALI A. HABASHI, POCATELLO, ID (US)

(21) Appl. No.: 17/122,364

(22) Filed: Dec. 15, 2020

Related U.S. Application Data
(60) Provisional application No. 62/952,528, filed on Dec. 23, 2019.

(51) Int. Cl. A61K 38/26; A61K 47/54

(52) U.S. Cl. CPC A61K 38/26; A61K 47/548

(57) ABSTRACT

GLP-1 agonist peptide conjugates, methods of forming the conjugates, and methods of using the conjugates are described. Peptide conjugates include a GLP-1 agonist peptide (e.g., GLP-1 or a functional equivalent thereof), a linking agent, and a targeting moiety. Linking agents can include polymeric linkers such as PEG linkers, e.g., monodisperse PEG linkers. Targeting moieties can target tissue or cell types. Targeting moieties can include sulfhydryl groups for targeting hydroxyapatite of bone tissue. Conjugates can exhibit extended plasma half-life and can target bone tissue for use as a reservoir for extended delivery of the peptide.

### CROSS REFERENCE TO RELATED APPLICATION

This application claims filing benefit of U.S. Provisional Patent Application Ser. No. 62/952,528, having a filing date Dec. 23, 2019, entitled “Bone-Targeted GLP-1 Agonists Conjugates for Sustained Glycemic Control in Type 1 and Type 2 Diabetes,” which is incorporated herein by reference in its entirety.

### SEQUENCE LISTING

This application contains a Sequence Listing which has been submitted electronically in ASCII format and is hereby incorporated by reference in its entirety. Said ASCII copy, created on Feb. 10, 2021, is named ISU-101_Sequence_List.txt and is 6,708 bytes in size.

### BACKGROUND

Diabetes mellitus occurs in two forms, both of which are marked by hyperglycemia: type 1, caused by autoimmune destruction of pancreatic beta cells, and type 2, which is a result of defective insulin secretion or insulin resistance. The incidence of type 1 diabetes varies from 0.8-1.7% in Europe and the United States, and mostly occurs in children and adolescents. A majority (90-95%) of adult onset diabetic patients have been diagnosed with type 2 diabetes. The incidence of type 2 diabetes has steadily increased over the past few decades, and the global prevalence is estimated to reach 300 million by 2025 (2008, Hays, NP, et al.).

The incretin system plays an important role in type 2 diabetes. This system produces incretin hormones such as glucagon-like peptide 1 (GLP-1) and is involved in stimulation of insulin secretion in response to oral intake of food. It has been reported that insulin secretion is higher in response to oral glucose ingestion as compared to insulin secretion in response to an isoglycemic intravenous glucose infusion, and this phenomenon has been attributed to effects of the incretin system (1964, Elrick H, et al.).

Recently, the incretin system has drawn the attention of the diabetic research community as an important target in the management of diabetes, and particularly, type 2 diabetes. In this research, two main and several shorter versions of incretin peptide hormones have been identified, including glucose-dependent insulinotropic polypeptide GIP), GLP-1, and dipeptidyl peptidase IV (DPP-4), as well as enzymatic degradation products of GLP-1, such as GLP-1 (7-37), GLP-1 (7-36), GLP-1 (9-36), etc. (2006, Drucker D J, et al.). GLP-1 and some of its shorter analogues have garnered particular attention for their glucose-lowering effects, ability for glucagon secretion inhibition, and gastric motility retention in diabetic patients (2007, Baggio L L). Diminished incretin effect has been attributed to downregulation of GLP-1 receptor and lower GLP-1 effect. As a result, insulin secretion capacity is impaired, insulin resistance develops, and hyperglycemia results. GLP-1 receptor stimulation by GLP-1 agonists has been shown to enhance insulin secretion in response to glucose intake. Several beneficial pharmacological effects have been reported for GLP-1 agonists including: lowering of glucagon plasma level, increase of insulin sensitivity, reduction of gastric motility, induction of satiety, lowering of A1C, lowering of free fatty acid level, and reduction of weight (2017, Hinnen, D).

Unfortunately, therapeutic application of native GLP-1 agonists has been hindered by their very short biological half-lives. Different approaches have been attempted to address this issue with limited success. For instance, chemical structure modification of the peptide and special formulation design resulted in a several-fold increase in their half-life and some short, intermediate, and long-acting products have been developed. However, these compounds are administered through subcutaneous injection in twice daily, daily, and weekly intervals, which is problematic in many respects.

What is needed in the art are methods and materials for delivery of GLP-1 agonists that can provide extended stimulation of GLP-1 receptor and improve the effect of the GLP-1 hormone so as to provide sustained glycemic control to subjects in need thereof.

### SUMMARY

According to one embodiment, disclosed is a peptide conjugate comprising a GLP-1 agonist peptide and a linking agent comprising a first end and a second end. The linking agent is bonded to the GLP-1 agonist peptide at the first end of the linking agent, and a targeting moiety is bonded to the linking agent at the second end of the linking agent. The targeting moiety can bind the peptide to a targeted biological material, e.g., a tissue or a cell type.

Also disclosed is a method for forming the peptide conjugate. For instance, a method can include reacting a primary amine of a GLP-1 agonist peptide with a first terminus of a linking agent and reacting a second terminus of the linking agent with a targeting moiety.

Also disclosed is a method for delivering a GLP-1 agonist peptide to an area. For instance, a method can include contacting an area with a peptide conjugate that comprises a GLP-1 agonist peptide, a linking agent bonded to the GLP-1 agonist peptide at a first end of the linking agent, and a targeting moiety bonded to the linking agent at a second end of the linking agent. The targeting moiety includes a functionality that binds a biological material (e.g., bone tissue) at the area following the contact.

### BRIEF DESCRIPTION OF THE FIGURES

A full and enabling disclosure of the present subject matter, including the best mode thereof to one of ordinary skill in the art, is set forth more particularly in the remainder of the specification, including reference to the accompanying figures in which:

FIG. 1 presents one exemplary conjugate and formation approach therefore as disclosed herein. FIG. 1 discloses SEQ ID NOs: 3, 6, and 7, respectively, in order of appearance.

FIG. 2 presents one exemplary conjugate and formation approach therefore as disclosed herein. FIG. 2 discloses SEQ ID NOs: 3, 8, and 9, respectively, in order of appearance.

FIG. 3 presents one exemplary conjugate and formation approach therefore as disclosed herein. FIG. 3 discloses SEQ ID NOs: 3, 10, and 5, respectively, in order of appearance.

FIG. 4 presents one exemplary conjugate and formation approach therefore as disclosed herein. FIG. 4 discloses SEQ ID NOs: 3, 11, and 12, respectively, in order of appearance.

FIG. 5 presents one exemplary conjugates and formation approach therefore as disclosed herein. FIG. 5 discloses SEQ ID NOs: 3, 13, and 14, respectively, in order of appearance.

FIG. 6 presents a chromatogram of a conjugate as described herein.

FIG. 7 presents the mass spectrometry results of a conjugate as described herein.

FIG. 8 presents the binding capacity of GLP-1 and a GLP-1 conjugate as described in various binding mediums. On the figure—*significantly different from GLP-1, p<0.01, [symbol] significantly different from PBS 50 mM, p<0.01, ¶ significantly different from PBS, p<0.01.

Repeat use of reference characters in the present specification and drawings is intended to represent the same or analogous features or elements of the present invention.

### DETAILED DESCRIPTION

Reference will now be made in detail to various embodiments of the disclosed subject matter, one or more examples of which are set forth below. Each embodiment is provided by way of explanation of the subject matter, not limitation thereof. In fact, it will be apparent to those skilled in the art that various modifications and variations may be made in the present disclosure without departing from the scope or spirit of the subject matter. For instance, features illustrated or described as part of one embodiment, may be used in another embodiment to yield a still further embodiment.

The present disclosure is generally directed to methods for utilizing biological target as a depot for retaining and release of GLP-1 agonists. In one embodiment, skeletal bone matrix can be utilized as a depot. Also disclosed are conjugates of GLP-1 agonists that can be targeted to a particular biological target that can function as a depot and provide sustained release of the GLP-1 agonist carried by the conjugate.

Disclosed methods can provide for improved release of GLP-1 agonists and thereby stimulation of the GLP-1 receptor which can lead to sustained glycemic control. Disclosed methods can eliminate or reduce the number of injections required in treatment of hypoglycemia, e.g., treatment of type 2 diabetes, which can increase patient compliance and adherence to a treatment protocol and produce improved therapeutic outcome. Disclosed methods can also improve the safety profile of a treatment. Oral formulations are also encompassed herein, which can exhibit stomach acid resistance for successful oral delivery. This approach can avoid invasive subcutaneous administration altogether.

The methods utilize a GLP-1 conjugate that has been designed to target a biological target of choice as a depot for a GLP-1 agonist that is also a component of the conjugate. In one embodiment, a conjugate can include a GLP-1 agonist with 50, 55, 60, 65, 70, 75, 80, 85, 90, 95, 96, 97, 98, or 99% homology to a reference sequence that is a native GLP-1 agonist peptide as is known in the art.

In one embodiment, a GLP-1 agonist of a conjugate can include the full 37 amino acid GLP-1 peptide, i.e., HDEFE RHAEG TFTSD VSSYL EGQAA KEFIA WLVKG RG (SEQ ID NO: 1). In other embodiments, enzymatic degradation products of GLP-1 that exhibit GLP-1 agonist activity can be utilized, including, without limitation, GLP-1 (7-37), i.e., HAEGT FTSDV SSYLE GQAAK EFIAW LVKGR G (SEQ ID NO: 2), GLP-1 (7-36), i.e., HAEGT FTSDV SSYLE GQAAK EFIAW LVKGR (SEQ ID NO: 3), or GLP-1 (9-36), i.e., EGTFT SDVSS YLEGQ AAKEF IAWLVK GR (SEQ ID NO: 4). Other GLP-1 agonists as known in the art can alternatively be incorporated in a conjugate. GLP-1 agonists as encompassed can include those described previously; for example, in US Patent Application Publication 2012/0148586 to Chou, et al., U.S. Pat. No. 9,764,003 to Jensen, and U.S. Pat. No. 9,714,277 to Bednark, all of which being incorporated herein by reference.

As used herein, the single letter abbreviation for a particular amino acid, its corresponding amino acid, and three letter abbreviation are as follows: A, alanine (Ala); C, cysteine (Cys); D, aspartic acid (Asp); E, glutamic acid (Glu); F, phenylalanine (Phe); G, glycine (Gly); H, histidine (His); I, isoleucine (Ile); K, lysine (Lys); L, leucine (Leu); M, methionine (Met); N, asparagine (Asn); P, proline (Pro); Q, glutamine (Gin); R, arginine (Arg); S, serine (Ser); T, threonine (Thr); V, valine (Val); W, tryptophan (Trp); and Y, tyrosine (Tyr).

In some embodiments, a GLP-1 agonist component of a conjugate can include a functional equivalent, analog, or derivative of a GLP-1 agonist peptide. For instance, a GLP-1 agonist component can include the full GLP-1 peptide (SEQ ID NO: 1), or a functional fragment thereof e.g., SEQ ID NOs: 2, 3, or 4 as described above that can include amino acid substitutions, deletions and/or insertions in the GLP-1 agonist. GLP-1 agonist peptide functional equivalents, analogues or derivatives can be made by altering the known amino acid sequences by substitutions, additions, and/or deletions. For example, one or more amino acid residues within the sequence of the full GLP-1 agonist peptide (SEQ ID NO: 1) can be substituted by another amino acid of a similar polarity, which acts as a functional equivalent, resulting in a silent alteration. Substitution for an amino acid within the sequence may be selected from other members of the class to which the amino acid belongs. For example, the positively charged (basic) amino acids include arginine, lysine, and histidine. The nonpolar (hydrophobic) amino acids include leucine, isoleucine, alanine, phenylalanine, valine, proline, tryptophan, and methionine. The uncharged polar amino acids include serine, threonine, cysteine, tyrosine, asparagine, and glutamine. The negatively charged (acid) amino acids include glutamic acid and aspartic acid. The amino acid glycine may be included in either the nonpolar amino acid family or the uncharged (neutral) polar amino acid family. Substitutions made within a family of amino acids are generally understood to be conservative substitutions.

A GLP-1 agonist peptide may be obtained by any method known to those skilled in the art, including synthetic and recombinant techniques. By way of example, synthetic formation techniques including, without limitation, exclusive solid phase synthesis, partial solid phase synthesis, fragment condensation, classical solution synthesis, or native-chemical ligation can be utilized.

In addition to a GLP-1 agonist, a conjugate also includes at least one targeting moiety that includes a functional group that can bind the conjugate to the biological material of interest (e.g., a tissue type). The targeting moiety is conjugated to the GLP-1 by use of a functional group (e.g., a thiol functional group) that can be a component of any suitable chemical species that can be covalently bonded to the GLP-1 agonist peptide of the conjugate thereby linking the GLP-1 agonist to the targeting moiety. In general, the targeting moiety can be bonded to the GLP-1 agonist by use of a linking agent, e.g., a biocompatible polymeric or non-polymeric linking agent such as a polyethylene glycol or other linear or branched biocompatible hydrocarbon polymer or monomer. A linking agent can generally have any suitable length or number average molecular weight so as to link the targeting moiety with the GLP-1 agonist and provide for binding of the conjugate to the targeted biological material without destruction of the GLP-1 agonist. In some embodiments, the GLP-1 agonist can exhibit desired activity while bonded to the linking agent of the conjugate. In some embodiments, the GLP-1 can be released from the conjugate following bonding of the targeting moiety to the target, and the GLP-1 agonist can exhibit activity of the free GLP-1 agonist following release (optionally in conjunction with exhibiting activity while bonded to the linking agent).

In one embodiment, a linking agent can be conjugated to the peptide in such a fashion so as to maintain the activity of the peptide while bound. For instance, the linking agent can be bonded to the peptide at the N-terminus of the GLP-1 agonist peptide and maintain peptide activity while bound. However, this is not a requirement of disclosed conjugates, and in some embodiments, the linking agent can be bonded to the GLP-1 agonist peptide and a different location or at multiple locations along the peptide. For instance, the linking agent can bond a GLP-1 agonist peptide via primary amine groups of amino acids on the peptide, e.g., via a bonding reaction with a terminal primary amine of one or more of arginine, lysine, asparagine, glutamine, or alanine present on the GLP-1 agonist. In those embodiments in which the GLP-1 agonist is bonded to the linking agent along the peptide length (i.e., in addition to or instead of at a terminus), the peptide can maintain activity while bonded as well as or alternatively can exhibit activity following release from the conjugate.

In one embodiment, the linking agent can include a linear or branched water-soluble and non-peptidic polymer. In some embodiments, the linking agent can be one or more of soluble in water, stable to heat, inert to many chemical agents, resistant to hydrolysis, and nontoxic. The linking agent can be biocompatible and, as such, can be capable of coexistence with living tissues or organisms. The linking agent can be non-immunogenic and, as such, is not known to produce an immune response in the body.

A polymeric linking agent can encompass any polymer that includes one or more reactive functional groups that allow for covalent bonding with the GLP-1 agonist peptide and a tissue targeting moiety. In one embodiment, the polymeric linking agent can include a poly (alkylene glycol), e.g., a linear poly (alkylene glycol) such as a linear poly (ethylene glycol) (PEG). A polymeric linking agent is not limited to PEG-based polymers, however, and in other embodiments, polymeric linking agents as are known in the art may alternatively be utilized, examples of which include, without limitation, dextran; water soluble polyamino acids; polyglutamic acid (PGA); polylactic acid (PLA); polylactic-co-glycolic (PLGA); poly(D,L-lactide-co-glycolide) (PLA/PLGA); poly (hydroxy alkyl methacrylamide); polyglycerol; poly (amidoamine) idoamine) (PAMAM); and polyethylenimine (PEI) that can optionally be functionalize to include suitable reactivity for covalent bonding to a GLP-1 agonist.

A polymeric linking agent is not particularly limited with regard to size. For instance, suitable PEG-based linking agents can include, without limitation, PEG(100), PEG(200), PEG(300), PEG(400), PEG (500), PEG(600), PEG(1000), PEG(1500), PEG(2000), PEG(3000), PEG(3350), PEG(4000), PEG(5000), PEG(6000), PEG(8000), and PEG(10000), as well as methoxy and ethoxy derivatives thereof, and any PEG having a molecular size within and inclusive of any of the above indicated molecular weights, as well as larger or smaller polymers. In one embodiment, a polymeric linking agent can include less than about 100 monomeric components; for instance, from about 5 to about 80 monomeric components in the polymer, such as from about 15 to about 50 monomeric components, or from about 20 to about 40 monomeric components, in some embodiments.

A linking agent is not limited to polymeric linking agent. For instance, a non-polymeric crosslinking agent can be incorporated in a GLP-1 agonist peptide conjugate as a linking agent between the GLP-1 agonist peptide and a tissue targeting moiety.

By way of example, exemplary non-polymeric linking agents can include sulfosuccinimidyl-4-(N-maleimidomethyl)cyclohexane-l-carboxylate (Sulfo-SMCC); m-maleimidobenzoyl-N-hydroxysulfosuccinimide ester (Sulfo-MBS); 3-Maleimidobenzoyl-N-hydroxysuccinimide ester (MBS); sulfosuccinimidyl 6-(3′-[2-pyridyldithio]-propionamido)hexanoate (Sulfo-LC-SPDP); N-succinimidyl(4-iodoacetyl)aminobenzoate (SIAB); maleimido butryloxy-succinimide ester (GMBS); N-(e-MaleimidoCaproyloxy)-N-HydroxySuccinimide ester (EMCS); succinimidyl-6-((iodoacetyl)amino)hexanoate (SIAX); Succinimidyl-4-(p-maleimidophenyl)butyrate (SMPB); succinimidyl-4-(((iodoacetyl)amino)methyl) cyclohexane-I-carboxylate (SIAC); p-nitrophenyl iodoacetate (NPIA), m-maleimidobenzoyl-N-hydroxysulfosuccinimide ester (Sulfo-MBS); N-(e-MaleimidoCaproyloxy)-N-HydroxySuccinimide ester (EMCS); p-nitrophenyl iodoacetate (NPIA); as well as derivatives thereof (e.g., Sulfo-SIAB, Sulfo-GMBS, Sulfo- EMCS, etc.), or any combination thereof.

In one embodiment, characteristics of the linking agent, e.g., the molecular weight of a polymer, etc., can be modified to control characteristics of the conjugate, as well as characteristics of the activity of the GLP-1 agonist peptide carried by the conjugate. For instance, modification of the length of a linear polymeric linking agent can be utilized to effect half-life of the conjugate and/or release profile of the peptide from the conjugate.

In one embodiment, the conjugate can incorporate a linking agent having a well-defined structure, with little or no variations between individual members (e.g., a single isomer), which can provide a route for formation of a well-defined conjugate at high purity, and thus, provide for tight control capability of the characteristics of the conjugate. In one embodiment, the linking agent can incorporate a polymer having a narrow molecular weight distribution, and in one embodiment, a monodisperse polymer, e.g., a monodisperse PEG, such as a monodisperse linear PEG.

In one embodiment, a narrow molecular weight polymeric spacer can be formed from free radical polymerization followed by separation via, e.g., size exclusion chromatography. In free radical polymerization processes, molecular weight distributions can be narrowly controlled for chains having molecular weights between about 200 and 1,200 daltons and above. Typically, far less than 50% of the polymers in a formation batch have exactly the targeted molecular weight. Narrower distribution may be achieved with size exclusion chromatography, which can result in a greater amount of the polymer, e.g., about 80% or more of PEG polymers having a targeted molecular weight.

In one embodiment, a monodisperse polymer can be utilized as a polymeric linking agent. For instance, monodisperse PEG containing up to about 50 ethylene oxide units is available in the retail market, including linear PEG, as well as branched structures that include from about 3 to about 9 monodisperse linear chains. In one embodiment, the polymeric linking agent can include a linear monodisperse PEG of from about 3 to about 48 ethylene oxide units, from about 5 to about 40 ethylene oxide units, from about 10 to about 35 ethylene oxide units, or from about 15 to about 30 ethylene oxide units, in some embodiments. For instance, a PEG-based linking agent can include 2, 4, 8, 10, 12, 15, 17, 19, 20, 23 25, 27, 30, 35, 40, or 45 ethylene oxide units, in some embodiments.

A linking agent can include one or more reactive functional groups that allow for covalent bonding with both the GLP-1 agonist peptide and with a targeting moiety. In one embodiment, the linking agent can include heterofunctionality, e.g., two different reactive functional groups—one designed for covalent bonding with the GLP-1 agonist peptide (e.g., at the N-terminus) and one designed for covalent bonding with the targeting moiety—but this is not a requirement of the invention, and in other embodiments, the linking agent can include the same reactive functionality for bonding to both components of a conjugate. Suitable bonding functionalities are not particularly limited, examples of which can include, without limitation, hydroxyl, active ester; active carbonate; acetal; aldehyde; aldehyde hydrate; alkyl or aryl sulfonate; halide; disulfide; alkenyl; acrylate; methacrylate; acrylamide; active sulfone; amine; hydrazide; thiol; carboxylic acid; isocyanate; isothiocyanate; maleimide; vinylsulfone; dithiopyridine; vinylpyridine; iodoacetamide; epoxide; glyoxal; dione; mesylate; tosylate; or tresylate. Reactive functionality can be incorporated by use of a biologically-based functional group, e.g., biotin functionality or the like.

Conjugation between the GLP-1 agonist peptide and the linking agent can be carried out according to any suitable chemistry, which can usually depend upon the particular functional groups utilized in the conjugation reaction, as is known to one of ordinary skill in the art. In embodiments in which one or more N-alpha amino acids of the peptide are protected, the conjugation reaction can form exclusively (or nearly exclusively, e.g., about 90% or greater) isomers in which the linking agent is bonded only to the N-alpha amino acids of choice, e.g., at the amino terminus of the GLP-1 agonist peptide.

At a second site the linking agent can include at least one moiety designed to target a biological target. In one embodiment, the targeting moiety can target a biological tissue. For instance, a targeting moiety can be a bone targeting moiety that includes a thiol functional group (—SH). However, it should be understood that the targeting moiety is not limited to bone tissue targeting, and the GLP-1 agonist peptide conjugates can include targeting moieties directed toward other tissues than bone. Moreover, the targeting moiety is not limited to targeting biological tissue of any particular type and can target one or more particular cell types, ECM components, etc. For example, in one embodiment, a GLP-1 agonist peptide conjugate can include a folate molecule (vitamin B9) that can target a folate receptor, or a small peptide binding element as is known in the art, e.g., Arg-Gly-Asp (RGD) as can be utilized to target αvβ3 motif of integrin, etc.

In one embodiment, the targeting moiety can include a thiol-containing component for targeting a component of bone tissue. In one particular embodiment, a bone targeting moiety can include a bisphosphonate component. As utilized herein, the term “bisphosphonate” includes any salts, solvates and/or hydrates of bisphosphonate structures as defined herein. For instance, a bone targeting moiety can be a bisphosphonate having the following general structure:

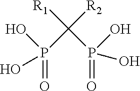

in which at least one of R₁ and R₂ includes a group that allows for covalent bonding with the linking agent. For example, R₁ and R₂ can be independently selected from hydrogen, substituted or unsubstituted alkyl, substituted or unsubstituted cycloalkyl, substituted or unsubstituted aryl, substituted or unsubstituted heteroaryl, substituted or unsubstituted heteroalicyclic, halo, hydroxy, thiol, alkoxy, thioalkoxy, aryloxy, thioaryloxy, amine, or alkylamine.

In some embodiments, at least one of R₁ and R₂ is hydroxy or hydrogen, and the other one can include a thioalkyl, alkylamine, or alkoxy. An alkyl chain of a bisphosphonate can be varied, variation of which can, in one embodiment, be utilized to modify and design desirable characteristics for a conjugate. For instance, in some embodiments, an alkyl or alkoxy chain of a bisphosphonate can have from 1 to about 10 carbon atoms in the backbone chain, or from 1 to about 6, or from about 2 to about 5 carbon atoms in some embodiments.

By way of example, in one embodiment, a bisphosphonate for inclusion on a peptide conjugate can have a structure of:

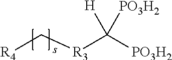

in which s is from 1 to 10, R₃ is selected from S, N, O, and R₄ includes a functional group that allows for covalent bonding with the linking agent, examples of which can include, without limitation, hydroxyl; active ester; active carbonate; acetal; aldehyde; aldehyde hydrate; alkyl or aryl sulfonate; halide; disulfide; alkenyl; acrylate; methacrylate; acrylamide; active sulfone; amine; hydrazide; thiol; carboxylic acid; isocyanate; isothiocyanate; maleimide; vinylsulfone; dithiopyridine; vinylpyridine; iodoacetamide; epoxide; glyoxal; dione; mesylate; tosylate; or tresylate.

Bisphosphonates are widely used for the treatment of osteoporosis and have also been used as a vehicle for delivering bone-targeted prodrugs to osseous tissue via the bisphosphonic moiety that binds hydroxyapatite. The bisphosphonate component of the GLP-1 agonist peptide conjugates described herein can thus be utilized in one embodiment for targeting bone tissue for utilizing bone tissue as a reservoir or depot for sustained release of the GLP-1 peptides carried by the conjugate. As such, in some embodiments, the GLP-1 agonist peptide conjugates can be utilized in therapies directed to treatment of hypoglycemia, e.g., type 1 and type 2 diabetes.

Conjugation between the targeting moiety and the linking agent can be carried out according to any suitable chemistry, which can usually depend upon the particular functional groups utilized in the conjugation reaction, as is known to one of ordinary skill in the art.

In one embodiment, a GLP-1 agonist peptide can be formed according to a solid phase peptide synthesis in which the N-alpha protecting groups can remain on the peptide throughout formation of the conjugate. In this embodiment, the protecting groups can be removed following formation of the conjugate, or alternatively, following conjugation between the linking agent and the conjugate and prior to conjugation with the bisphosphonate component. Deprotection of the protecting groups can be carried out according to any suitable methodology, for instance, by use of a moderately strong acid (e.g., trifluoroacetic acid) when utilizing Boc as a protecting group or by use of a mild base (e.g., piperidine) when utilizing Fmoc as a protecting group. In other embodiments, a portion or all of the N-alpha protecting groups can be removed throughout formation of the conjugate, and multiple linking agents can be conjugated to the GLP-1 agonist peptide. As such, multiple tarting moieties can likewise be conjugated to the GLP-1 agonist peptide.

Formation methods as may be utilized in preparing the disclosed conjugates can provide a GLP-1 agonist peptide conjugate at high purity. For instance, through utilization of solid phase synthesis peptide formation techniques that provide tight control over the presence and location of N-alpha protecting groups during formation of the conjugate, the conjugation reaction between a GLP-1 agonist peptide and the linking compound can be targeted to only particular amino acid residues of the peptide, e.g., only the N-terminus of the peptide or only select residues (e.g., one or more lysine terminal amines) of the peptide, and formation of multiple isomers can be prevented.

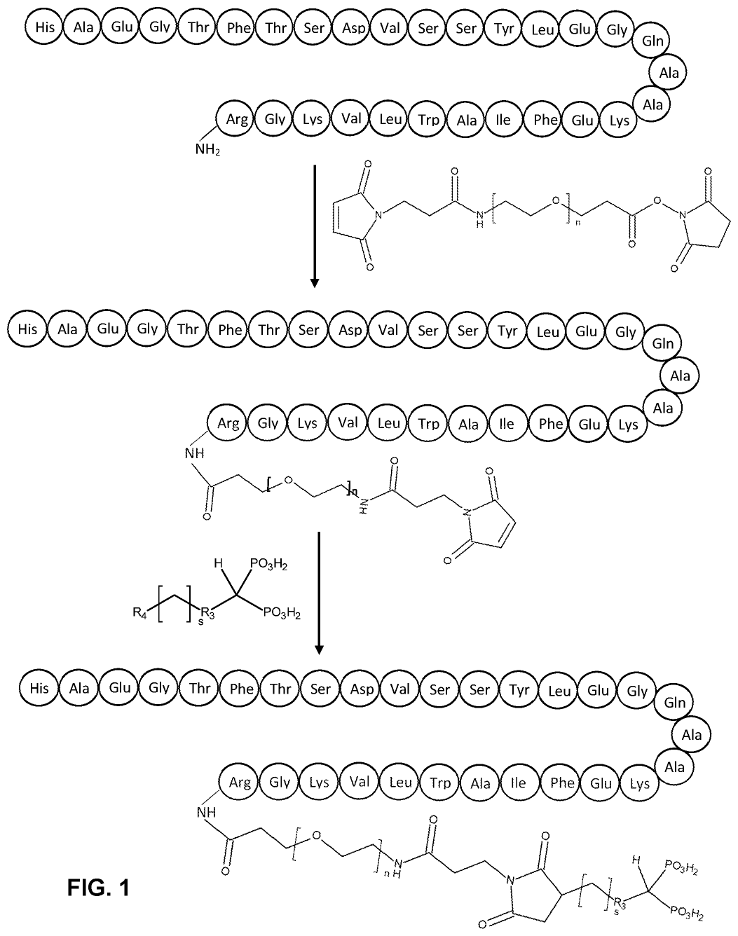

In one embodiment, a conjugate can be formed according to a conjugation reaction that results in formation of a plurality of different isomers. For example, as the GLP-1 chemical structure has three primary amines (the terminal amine, Lys²⁶, and Lys³⁴), a conjugation reaction between a linking agent and primary amines can form a number of different mono-, di-, and tri-conjugated isomers (e.g., multiple different isomers as illustrated in FIG. 1-5). Following formation, the isomers can then be separated, for instance by use of an analytical reverse-phase HPLC with UV-VIS detector, and collected of eluted major peaks by use of a fraction collector. Such a conjugation and separation strategy can be used in one embodiment to form a high purity product with a targeted site conjugation. Such an embodiment can also be utilized to simplify the identification of lead isomers; for instance, in particular drug development.

Following formation and separation, the targeting characteristics of the isolated conjugate isomers can be tested and compared. For instance, a bone-targeting moiety of the different isomers can be tested using hydroxyapatite binding assay and in-vitro bioactivity can be compared with native GLP-1, which can be assessed towards the GLP receptor utilizing a cell culture assay followed by cyclic AMP measurements of receptor activation.

A formation and product development process can include examination of each separated isomer and determination of differences in desirable activity between the different isomers. Such determination can be carried out in one embodiment by digesting each isolated conjugate using a protease enzyme (e.g., trypsin) and characterization of the fragments containing the targeting moiety by MALDI-ToF. For instance, the mass spectrum of blank matrix, native GLP-1 agonists, and different conjugate isomers can be compared to determine the site of conjugation. The result can be confirmed with NMR and IR spectroscopy, and additionally, with Light Scattering and Circular Dichroism techniques.

Based on the results of such studies, a pure product of desirable potent isomers can be manufactured, which can improve optimization of lead compounds for the conjugates, as well as fulfill the regulatory mandated Good Laboratory Practice toxicology studies.

After confirmation of final chemical structure of active conjugates, pharmacokinetic and toxicology studies can be conducted using a suitable animal model for calculation of biological half-life and other PK parameters in comparison with native peptides. The tissue distribution of conjugates can then be conducted on main organs, such as bone (major target), heart, liver, kidney, long, etc. For pharmacodynamic studies, animal models of diabetes can be utilized, and based on the calculated half-life, the optimal dose and dosing interval can be studied, and as primary outcome, plasma glucose level can be recorded.

Through a separation technique as described to obtain high purity single isomers combined with utilization of a linking agent having a single isomer and/or a narrow molecular weight range, or, in one embodiment, a monodisperse polymeric linking agent as discussed previously, a GLP-1 agonist peptide conjugate structure can be even more narrowly controlled, and as such, can exhibit well-defined characteristics. For instance, in one embodiment, a formation method can provide a monodisperse single isomer of a GLP-1 agonist peptide conjugate at a high purity of about 90% or higher.

The conjugation of a GLP-1 agonist peptide with a targeting moiety can be utilized to target a GLP-1 agonist peptide to tissue and utilize the tissue as a reservoir for sustaining therapeutic plasma levels of the active peptide either systemically or in a targeted area. Moreover, the conjugate can provide for high stability in solution and prolongation of plasma half-life of the GLP-1 agonist peptide as compared to the native peptide. For instance, a GLP-1 agonist peptide conjugate as described herein can exhibit a plasma half-life of about 60 minutes or more, about 120 minutes or more, about 200 minutes or more, about 300 minutes or more, or about 400 minutes or more, in some embodiments. For example, a GLP-1 agonist peptide conjugate can exhibit an increase, e.g., a 2 or more-fold increase, a 3 or more-fold increase, a 4 or more-fold increase, or even higher in some embodiments in plasma half-life after intravenous or subcutaneous injection as compared to the native peptide, which can result in longer mean resistance time for the GLP-1 agonist peptide of the conjugate compared with that of the native peptide alone.

A GLP-1 agonist peptide conjugate may be delivered or administered acutely or chronically according to various delivery methods, including intravenous delivery, oral delivery, osmotic pumps, and so forth.

Compositions for parenteral delivery, e.g., via injection, can include pharmaceutically acceptable aqueous and nonaqueous carriers, diluents, solvents, or vehicles such as, without limitation, water, ethanol, polyols (e.g., glycerol, propylene glycol, polyethylene glycol and the like), carboxymethylcellulose and suitable mixtures thereof, vegetable oils (e.g., olive oil) and injectable organic esters such as ethyl oleate. In addition, the composition can contain minor amounts of auxiliary substances such as wetting or emulsifying agents, pH buffering agents and the like that can enhance the effectiveness of the biologically active compound. These compositions may also contain adjuvants such as preservatives, wetting agents, emulsifying agents, and dispersing agents.

Prevention of the action of microorganisms may be ensured by the inclusion of various antibacterial and antifungal agents such as paraben, chlorobutanol, phenol, sorbic acid and the like. It may also be desirable to include isotonic agents such as sugars, sodium chloride and the like.

A composition can include one or more oil-soluble antioxidants including, without limitation, butylated hydroxytoluene (BHT), ascorbyl palmitate, butylated hydroxyanisole (BHA), a-tocopherol, phenyl-a-naphthylamine, hydroquinone, propyl gallate, nordihydroguaiaretic acid, and mixtures thereof, as well as any other known oil-soluble antioxidant compatible with the other components of the compositions. Mineral oils, animal oils, vegetable oils, and silicones can be incorporated in topical creams or lotions as disclosed herein. In addition to such oils, other emollients and surface active agents can be incorporated in an emulsion.

A composition may also contain, as optional additions, one or more soluble or dispersible pharmaceutically acceptable ingredients generally used in pharmaceutical compositions. Typical such ingredients include, for example, a preservative or antioxidant such as methyl or propyl paraben, imidazolidinyl urea and the like; a water or oil soluble vitamin such as vitamin C, tocopheryl linoleate and the like; and/or a colorant, odorant, humectant, thickener, and the like. In general, from about 0.1 to about 15 percent total weight of such optional additives may be incorporated into a composition.

A composition may be made into a wide variety of product forms suitable for, e.g., administration via oral or parenteral administration. Each additive of a composition may generally constitute between about 0.05% to about 15% of the total weight of the formulation. In one embodiment, a composition can include an additive in an amount between about 0.05% and about 10% or between about 0.05% and about 8%, or between about 0.05% and about 7%, or between about 0.05% and about 6%, or between about 0.05% and about 5% of the total weight of the formulation.

The present invention may be better understood with reference to the Examples, set for below.

### EXAMPLE 1

FIG. 1-FIG. 5 illustrate examples of GLP-1 agonist peptide conjugates as described herein. In these embodiments, the linking agent included a bi-functional PEG-based polymer that included a maleimide functionality and N-hydroxysuccinimide functionality for linking a targeting moiety with the GLP-1 agonist peptide.

These bone seeking bisphosphonate (BP)-mediated conjugates were prepared by PEGylating of a GLP-1 agonist peptide with bifunctional Maleimide-PEG-NHS (Mal-PEG-NHS).

In a first step, 200 μL of a GLP-1 agonist peptide solution (10 mg/mL in DMSO) was mixed with 100 μL of Mal-PEG-NHS (50 mg/mL, in DMSO) at room temperature for 1 hour while rotating gently.

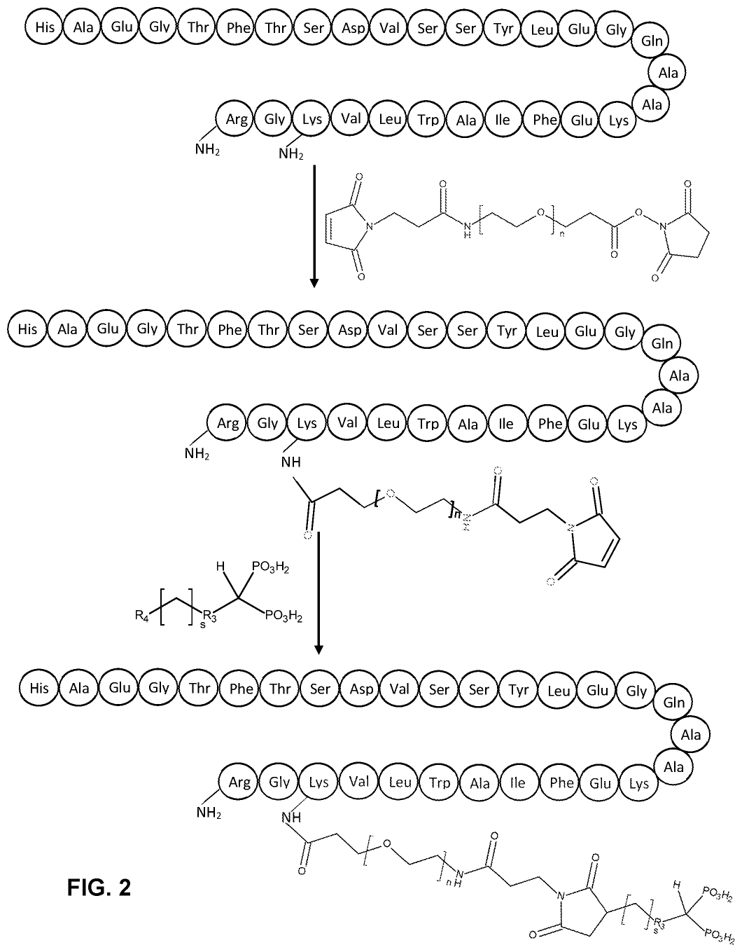

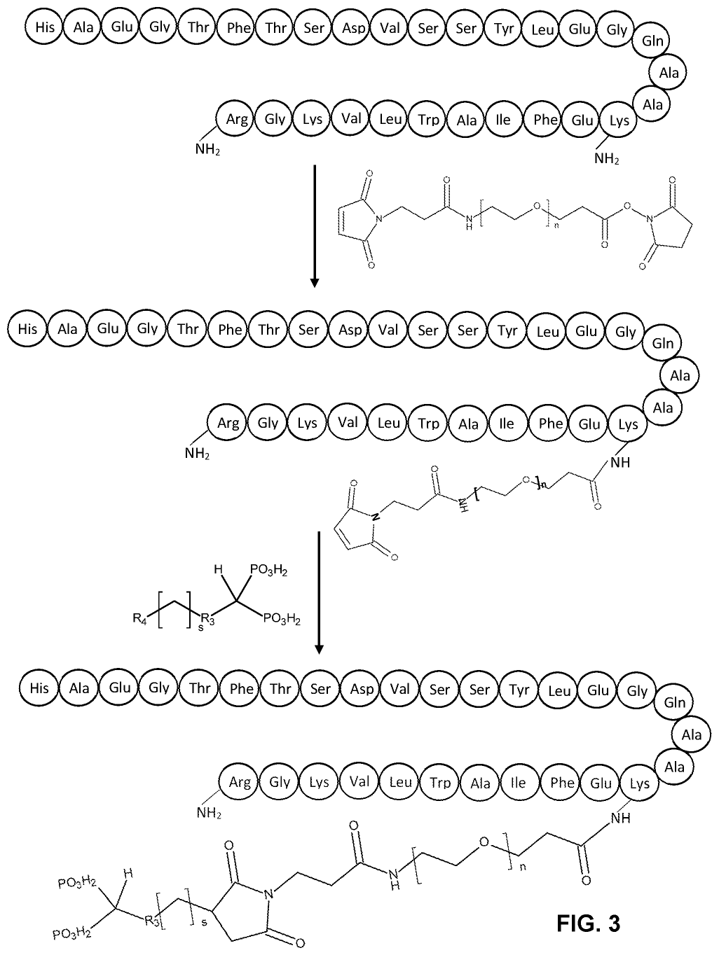

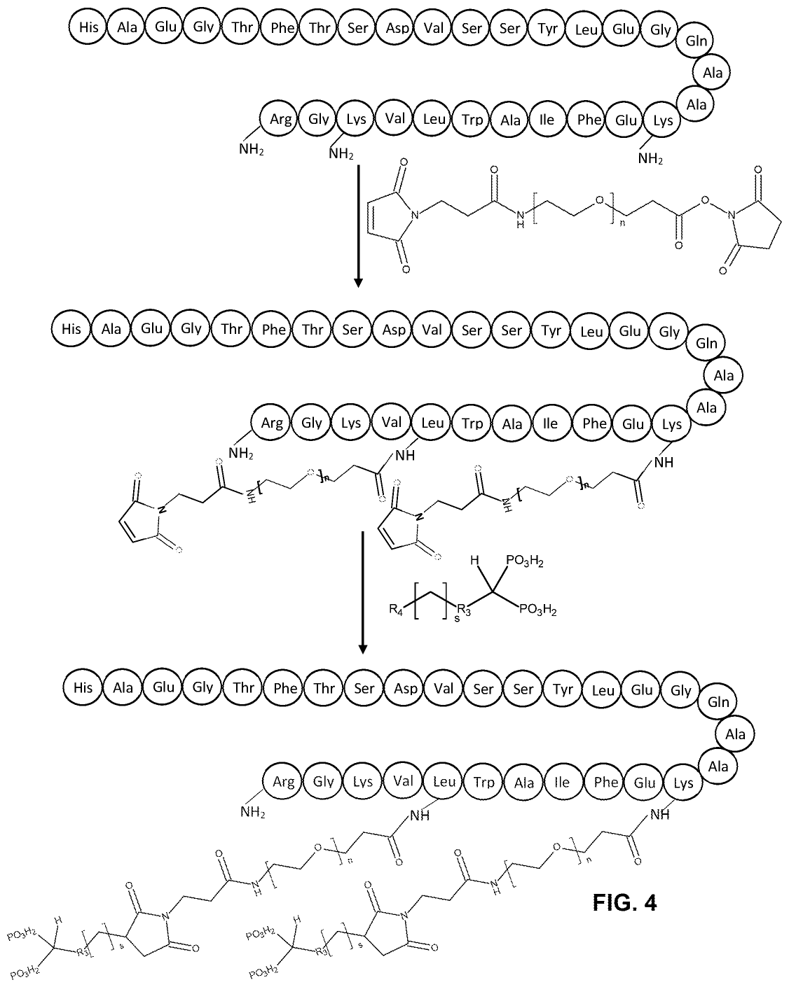

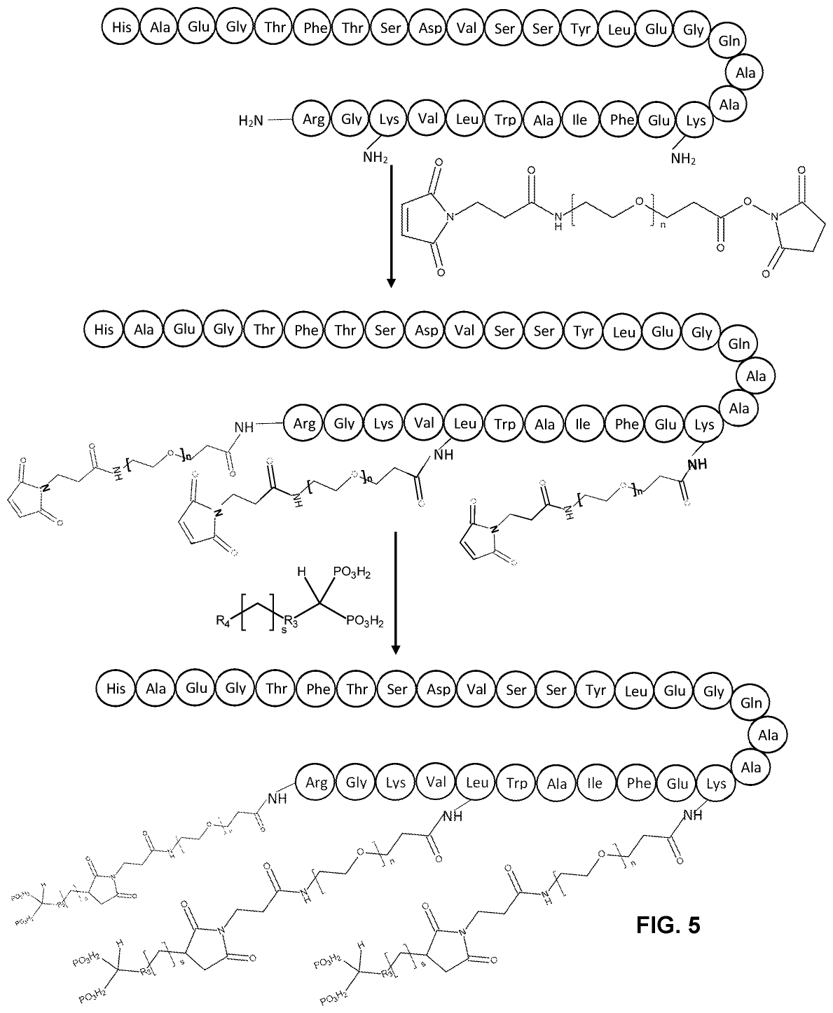

In a second step, to the mixer was added 2 mL of Thiol-BP solution (25 mg/mL in phosphate buffer, 100 mM, pH 7.4). Depending on the chemical structure of peptides and number of Lys amino acid protective groups in their sequence, this reaction resulted in mono-substituted GLP-PEG-BP conjugates (FIG. 1, FIG. 2, FIG. 3), di-substituted GLP-PEG-BP conjugate (FIG. 4) or tri-substituted GLP-PEG-BP conjugate (FIG. 5).

The final solution was dialyzed to remove unreacted components.

After confirmation of the conjugate formation, for instance, using MALDI-ToF, the resulting isomers, as illustrated in FIGS. 1-5, were separated using an analytical reverse-phase HPLC with UV-VIS detector and eluted major peaks were collected by use of a fraction collector. Products were then freeze-dried overnight. The resulting powders were analyzed by use of MALDI-ToF to confirm their molecular mass and other characteristics.

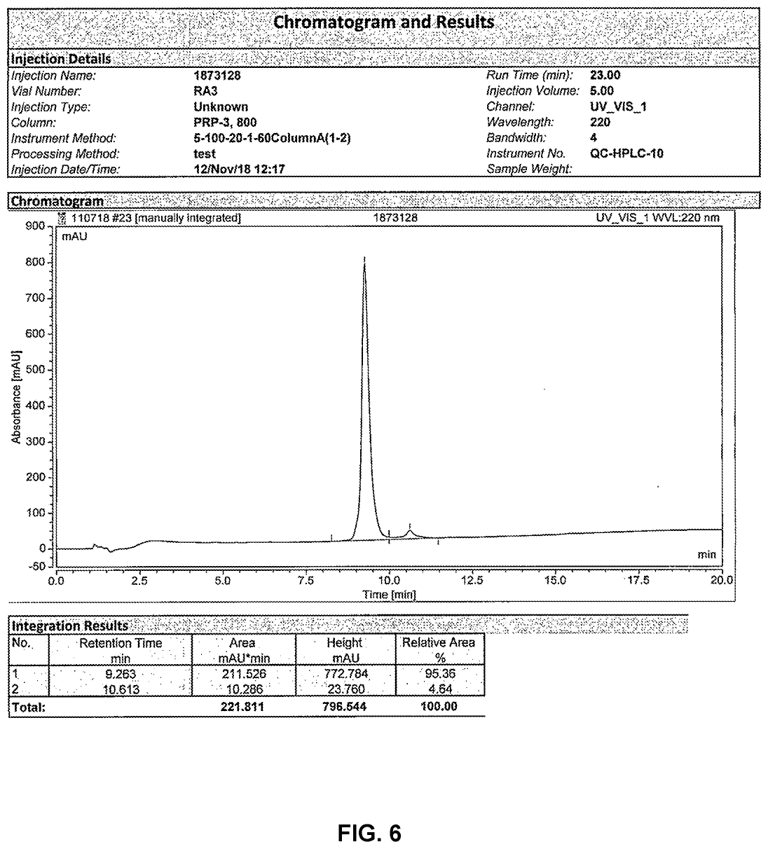

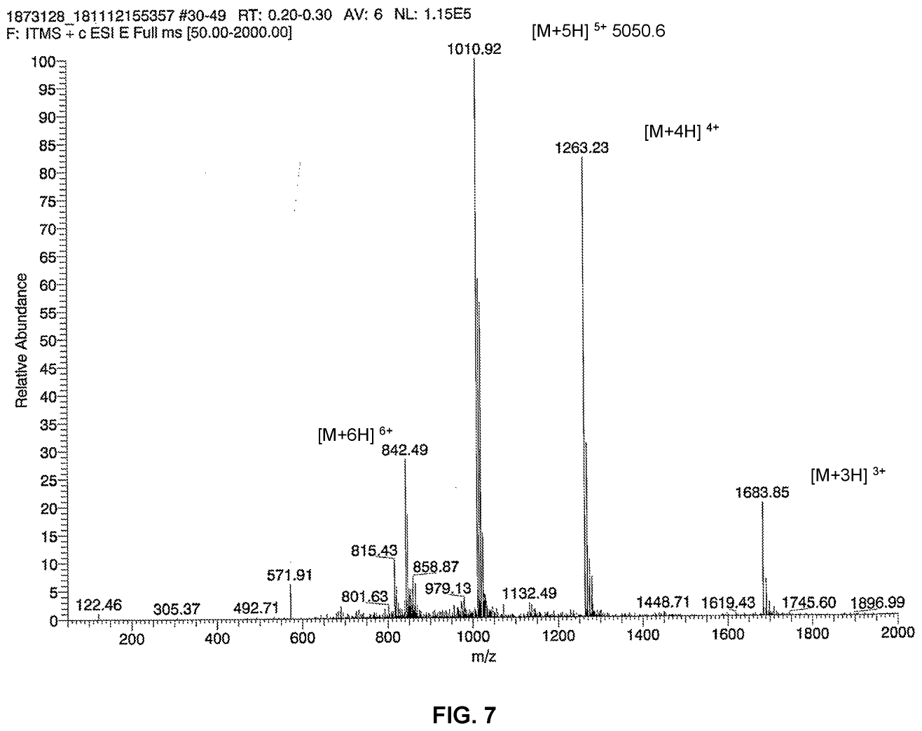

FIG. 6 and FIG. 7 present HPLC and MS data, respectively, for a conjugate as disclosed herein. The conjugate included a GLP-1 (7-36) peptide (SEQ ID NO: 3) with the linking agent conjugated to the bisphosphonate targeting moiety at the Lys²⁶ as illustrated in FIG. 3 and thus had the following sequence:

H-HAE GTF TSD VSS YLE GQA A-K(Mal-PEGₙ-BP)-E FIA WLV KGR —NH₂ (SEQ ID NO: 5)

Table 1 and Table 2, below, summarizes several characteristics of the conjugate.

TABLE 1

| Physicochemical | | |
|---|---|---|
| Properties | Regular AA Properties | |
| 1A 280 [mg/ml]* 0.7 | Charged AA | Polar AA |
| | H, E, D, K, R 7 | H, E, T, S, D, Q, K, R 13 |
| Charged at pH 7* −1.0 | Acid AA E, D 4 | Hydrophobic AA |
| | | A, G, F, V, Y, L, I, W 16 |
| Isoelectric Point* 4.6 | Basic AA H, K, R 3 | |

*theoretical values

TABLE 2
QC Data

| Attribute | Test Method | Acceptance Criteria | Result |
|---|---|---|---|
| Appearance | Visual | Report Result | White Powder |
| % Peak Area by HPLC | HPLC | ≥90% | 95% |
| Identity | MS | 5050.6 ± 0.2% | 5050.6 |

### EXAMPLE 2

To evaluate the bone mineral affinity of the GLP-1 conjugate described in Example 1, hydroxyapatite (HA) binding in vitro studies were conducted. Briefly, 10 μg of GLP-1 or equivalent amount of GLP-1 conjugate was mixed with 5 mg of HA powder in 500 μL of the binding buffer with various concentrations (double-distilled water (D.D.), 10 mM PBS [pH 7.4], 50 mM PBS [pH 7.4], acetate buffer [pH 4]) . Similarly, tubes containing an equivalent amount of GLP-1 or GLP-1 conjugate in corresponding buffers without HA were used as a negative control. Mixtures were shaken gently at room temperature for 1 hour and then were centrifuged at 10,000 g for 5 minutes. The supernatant was separated and assayed for unbound drug, using a fluorescence spectrometer (λEx 215 nm, λEm 305 nm). The percentage of HA binding was calculated as:

(Intensity of control−the intensity of supernatants)/Intensity of control×100%

Each experiment was measured in triplicate.

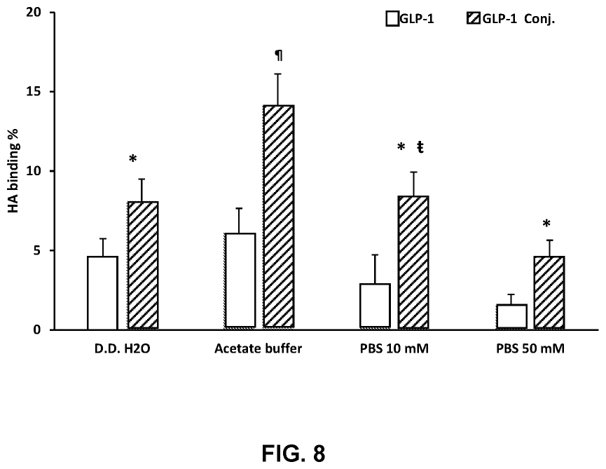

The results are illustrated in FIG. 8, which indicates that the GLP-1 conjugate exhibited significantly different binding capacity to HA in various medium when compared with plain GLP-1 peptide (p<0.01). The binding capacity was impacted by the pH and phosphate ion concentration. The acidic pH (Acetate buffer pH 4 vs PBS pH 7.4) and lower buffer concentration (10 mM vs 50 mM) resulted in higher binding.

While certain embodiments of the disclosed subject matter have been described using specific terms, such description is for illustrative purposes only, and it is to be understood that changes and variations may be made without departing from the spirit or scope of the subject matter.

What is claimed is:

1. A peptide conjugate comprising a GLP-1 agonist peptide, a linking agent comprising a first end and a second end, the linking agent being bonded to the GLP-1 agonist peptide at the first end of the linking agent, and a targeting moiety bonded to the linking agent at the second end of the linking agent.

2. The peptide conjugate of claim 1, wherein in the GLP-1 agonist peptide comprises SEQ ID NO: 1 or a peptide analogue or functional fragment of SEQ ID NO: 1.

3. The peptide conjugate of claim 1, wherein the GLP-1 agonist peptide comprises SEQ ID NO: 2, SEQ ID NO: 3, or SEQ ID NO: 4.

4. The peptide conjugate of claim 1, wherein the linking agent comprises a polymeric linking agent.

5. The peptide conjugate of claim 4, wherein the polymeric linking agent comprises a poly (ethylene glycol).

6. The peptide conjugate of claim 5, wherein the poly (ethylene glycol) is a monodisperse poly (ethylene glycol).

7. The peptide conjugate of claim 4, wherein about 80% or more of the polymeric linking agent has an identical molecular weight.

8. The peptide conjugate of claim 1, wherein the targeting moiety comprises a sulfhydryl group.

9. The peptide conjugate of claim 8, wherein the targeting moiety comprises a bisphosphonate.

10. The peptide conjugate of claim 9, wherein the bisphosphonate has a structure of: 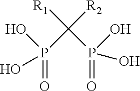
in which R₁ and R₂ are independently selected from hydrogen, substituted or unsubstituted alkyl, substituted or unsubstituted cycloalkyl, substituted or unsubstituted aryl, substituted or unsubstituted heteroaryl, substituted or unsubstituted heteroalicyclic, halo, hydroxy, thiol, alkoxy, thioalkoxy, aryloxy, thioaryloxy, amine, or alkylamine.

11. The peptide conjugate of claim 9, in which R₁ is hydroxy or hydrogen and R₂ is a thioalkyl, alkylamine, or alkoxy including an alkyl chain of from 1 to about 10 carbon atoms in length.

12. A method of forming a peptide conjugate comprising:
reacting primary amine of a GLP-1 agonist peptide with a first terminus of a linking agent; and
reacting a second terminus of the linking agent with a targeting moiety.

13. The method of claim 12, wherein the first terminus of the linking agent is reacted with multiple primary amines of the GLP-1 agonist peptide.

14. The method of claim 13, wherein the multiple primary amines comprise a terminal amine of the GLP-1 agonist peptide and amines of one or more lysine residues of the GLP-1 agonist peptide.

15. The method of claim 13, further comprising separating isomers of the peptide conjugate from one another.

16. The method of claim 15, wherein the separation provides a single isomer of the peptide conjugate at a purity of about 90% or greater.

17. The method of claim 12, wherein the GLP-1 agonist peptide comprises SEQ ID NO: 1, SEQ ID NO: 2, SEQ ID NO: 3, or SEQ ID NO: 4.

18. A method for delivering a GLP-1 agonist peptide to an area comprising contacting the area with a peptide conjugate that comprises a GLP-1 agonist peptide, a linking agent bonded to the GLP-1 agonist peptide at a first end of the linking agent, and a targeting moiety bonded to the linking agent at a second end of the linking agent, the targeting moiety comprising a functionality that binds a biological material at the area following the contact.

19. The method of claim 18, wherein the biological material comprises bone tissue.

20. The method of claim 18, wherein the peptide conjugate exhibits a half-life in the area of about 300 minutes or more.
